(19) United States
(12) Patent Application Publication
(10) Pub. No.: US 2022/0010351 A1
(43) Pub. Date: Jan. 13, 2022

(54) MICROBIAL CYTOMETRIC MOCK COMMUNITIES AND USE THEREOF AS STANDARD IN FLOW CYTOMETRY

(71) Applicants: HELMHOLTZ-ZENTRUM FÜR UMWELTFORSCHUNG GMBH - UFZ, Leipzing (DE); LEIBNIZ-INSTITUT DSMZ-DEUTSCHE-SAMMLUNG VON MIKROORGANISMEN UND ZELLKUL TUREN GMBH, Braunschweig (DE)

(72) Inventors: Susann MÜLLER, Leipzig (DE); Nicolas CICHOCKI, Libercourt (FR); Thomas HÜBSCHMANN, Weimar (DE); Jörg OVERMANN, Wolfenbüttel (DE)

(21) Appl. No.: 17/295,847

(22) PCT Filed: Nov. 29, 2018
(86) PCT No.: PCT/EP2018/082966

§ 371 Date: May 20, 2021

(51) Int. Cl. C12Q 1/06; G01N 15/14

(52) U.S. Cl. CPC C12Q 1/06; G01N 15/1434; G01N 2496/00; G01N 2015/1006; G01N 2333/305; G01N 2333/195; G01N 2333/245

(57) ABSTRACT

The present invention is directed to a microbial Cytometric Mock Community for use in flow cytometric analysis, the microbial Cytometric Mock Community comprising or consisting of cells of at least three different microbial species in a pre-defined ratio, wherein the at least three different microbial species are selected such that, when measured using flow cytometry, the specific gate pattern of each microbial species differs significantly from the specific gate pattern of the other microbial species of the microbial Cytometric Mock Community, preferably the at least three different microbial species differ in relative DNA content, relative genomic GC-content, relative cell size, Gram +/− affiliation and/or capacity to form spores. The microbial Cytometric Mock Community shall serve as standardization means that will help ecologists, microbiologists, molecular biologists and flow cytometrists to work on a standardized basis to allow comparison and exchange of data.

### BACKGROUND

Standards help to verify lab-workflows and ensure consistency of produced and processed data.

Flow cytometry allows the characterization of single cells and cell communities based on intrinsic and extrinsic properties. Intrinsic properties refer to properties and characteristics of the cells that can be measured without further additional means and include e.g. information about cell size, measured via light scatter, namely forward scatter: FSC, as well as cell density, measured via light scatter, namely side scatter: SSC, and auto-fluorescence. Extrinsic properties of cells denote properties and characteristics of the cells that are rendered measurable using additional means like e.g. specific or non-specific cell markers.

In general flow cytometry can be used to study pure microbial cultures in its physiological states and to determine intrinsic or extrinsic heterogeneities within a population. However, flow cytometry can also be used to study artificial and natural (biological) microbial communities comprising a multitude of different populations, strains and species of microorganisms. In this case, again intrinsic and extrinsic properties can be used for measurement and description of heterogeneities. However, calibration and verification of such methods are difficult. In particular, naturally occurring (biological) mixed populations or complex compositions of microorganisms comprise, depending on origin, 80 to 90% microorganisms which cannot easily be cultured which makes it difficult to calibrate and verify flow cytometric measurement of such samples.

Analysis of cell samples by flow cytometry is widespread in medical diagnostics and research on human cells, but it is also used for the measurement of the smaller cells of microbial populations. However, the cytometric analysis of microorganisms holds some surprises in tow. Firstly, unlike human cells which are frequently classified by specific fluorescently labelled markers (i.e. via antibodies), bacteria from natural communities cannot be highlighted in this way since exclusive markers are missing. Secondly, bacteria change their scatter behavior during growth and can increase or decrease their volume manifold in time ranges of minutes. Therefore, it is not straight forward to use light scatter as reliable parameter in characterizing specific microbial cell types. Thirdly, the number of different phylotypes in a sample can be as high as 10⁴, the differences of which cannot be visualized easily by cytometric techniques.

In addition to these variables originating from the sample origin, there are also technical device-related calibrations that need to be set and noise of different sources that needs to be controlled. For instance, the accuracy in alignment of the optics and the lasers of the cytometric device and their stability over time must be ensured. Photomultipliers are frequently sensitive to laboratory temperature, air humidity and air pressure and, thus, can affect signal intensities and influence instrumental noise. Hydrodynamics can fluctuate and particles in the sheath and tubes can cause unwanted background signal.

To align cytometers before measurement, to avoid false positive signals, and to compare samples between different measurement days, standardized mono-disperse beads are routinely used to calibrate flow cytometers. While this type of calibration may help to minimize potential technical bias in the system, said beads do not compensate for possible distortions of biological samples or microbial communities such as inadequate cell fixation, cell staining, or cell destruction due to rough sample handling by e.g. sonication or extensive washing steps. Each procedure of sample collection and sample conservation over time impacts the outcome of cytometric data. Almost all workflow steps can encompass pitfalls that cause inaccuracy of results impacting reliability or the reproducibility of an experiment.

Therefore, in addition to bead standards there is a need for further means that allow for standardization of flow cytometric measurement of complex samples like e.g. biological samples or microbial communities.

### DESCRIPTION OF THE INVENTION

The present invention is directed to the provision of a microbial Cytometric Mock Community for use in flow cytometry as defined in the claims. The microbial Cytometric Mock Community for use in flow cytometric analysis according to the present invention comprises or consists of cells of at least three different microbial species in a (constant) pre-defined ratio to each other, wherein the at least three different microbial species are selected such that, when measured using a flow cytometer, the specific gate pattern of each microbial species differs significantly from the specific gate pattern of the other microbial species of the microbial Cytometric Mock Community.

The microbial Cytometric Mock Communities of the present invention allow proper optical and fluid-dynamic calibration of a flow cytometer for measurement of microbial communities.

In most cases, flow cytometry is used for analysis of cells originating from human or animals. However, cells of such organisms usually exhibit an average cell size that is at least 10-times and an average cell volume that is at least 1000-times larger than the average cell size and average cell volume of cells of microbial species like e.g. bacterial species. Prokaryotic cells commonly will be located among instrumental noise of a flow cytometer that is calibrated for measurement of cells of human or animal origin. The microbial Cytometric Mock Community of the present invention allows for standardized calibration of a flow cytometer so that samples containing microorganisms can be measured in a reliable and reproducible manner. Measurements performed at different points in time and/or on different flow cytometric devices can be compared with each other provided both sets of data have been acquired using the microbial Cytometric Mock Community of the present invention for calibration and/or standardization.

In contrast to mono-dispersed beads currently available for calibration of flow cytometers for use in measurement of microbial samples, the microbial Cytometric Mock Communities of the present invention have the advantage that these microbial Cytometric Mock Communities allow also for control and verification of preparation and processing of the samples prior to measurement. Since flow cytometry is a very sensitive technique, it is well known in the art that the different steps of sample recovery and processing can have a significant impact on quality and quantity of the signal acquired. Thus, use of the microbial Cytometric Mock Community of the present invention allows for verification, standardization and/or control of at least one of the following steps:
fixation of cells;
sample preparation including washing steps and adjustment of optical density;
staining of the sample or cells;
calibration of flow cytometer;
stability of flow cytometric measurement over time; and
cell sorting/segregated gate allocation.

The microbial Cytometric Mock Community of the invention is intended for use in flow cytometry measurement and analysis. Flow cytometers are analytical devices which enable the characterization of particles like e.g. cells on the basis of optical parameters such as light scatter and fluorescence. In a flow cytometer, such particles or cells in a fluid suspension are passed by a detection region in which the particles or cells are exposed to an excitation light, typically from one or more lasers, and the light scattering and fluorescence properties of the particles/cells are measured. The parameters measured using a flow cytometer typically include the excitation light that is scattered by the particle/cell along a mostly forward direction, referred to as forward scatter (FSC), the excitation light that is scattered by the particle/cell in a mostly sideways direction, referred to as side scatter (SSC), and the light emitted from fluorescent molecules in one or more channels of the cytometric evaluation platform. Different cell types can be identified by the scatter parameters and the fluorescence emissions resulting from staining of the cells with certain staining agents. The data obtained from analysis of cells by flow cytometry are multidimensional (at least two-dimensional), wherein each cell corresponds to a point in a multidimensional space defined by the parameters measured. Groups or populations of cells form clusters of points in the data space. Such clusters may be defined in a subset of the dimensions, e.g., with respect to a subset of measured parameters, which correspond to populations that may differ in only one of the measured parameters. The identification of such clusters and, thereby, populations of cells can be carried out manually by drawing a gate around a cluster of data points displayed in one or more two-dimensional plots, referred to as “scatter plots” or “dot plots” of the data. Alternatively, clusters can be identified, and gates that define the limits of the clusters, can be determined automatically. Thus, the term “gate” generally refers to a set of boundary points identifying a subset of data of interest and, thereby, defining a cluster. The term “gating” generally refers to the process of defining a gate for a given set of data or cluster. The person skilled in the art is well aware of methods and techniques of gating a given set of data in order to arrive at a meaningful set of gates describing a sample or measurement result.

The cells of a microbial species, when measured using flow cytometry, can result in a group of separate populations which are provided as individual clusters in the data space of the measured parameters and, thus, lead to a set of gates, or a so called gate pattern, which describes the cells of this specific microbial species. Thus, the data set measured for cells of a given microbial species can be transferred into a gate pattern that is specific for said microbial species. The gate patterns of different microbial species may overlap to the extent that cells of a first microbial species are allocated to a gate already identified for a population of cells of a second microbial species. For the purpose of the present invention, the gate patterns of two microbial species are defined to differ significantly if the two specific gate patterns do not overlap more than a pre-defined threshold value. The threshold value defining the maximum degree of accepted overlap, however, can be defined prior to each new experiment. Preferably, a threshold value of 10% or less is used. In other words, if not more than 10% of the cells of the first microbial species are located in a gate assigned to the second microbial species and the other way round, the gate patterns of the two microbial species are held to differ significantly.

The microbial Cytometric Mock Community of the present invention comprises cells of at least three different microorganisms. When a microbial Cytometric Mock Community of the invention is measured using flow cytometry, the resulting data can be gated into a gate template comprising the specific gates and gate patterns of the cells of the at least three different microorganisms. In other words, the gate template of the microbial Cytometric Mock Community of the invention comprises the gate patterns of all the different microbial species encompassed by said microbial Cytometric Mock Community. Such a gate template, when designed and determined, can be stored and used at a later point in time for calibration and verification of flow cytometric measurement and analysis.

The microbial Cytometric Mock Community of the present invention comprises or consists of cells of at least three different microbial species. As used herein, the term “microorganism” is used in its art recognized meaning and includes prokaryotic and eukaryotic microbial species from the domains Archaea, Bacteria and Eukarya, the latter including yeast, fungi, protozoa, algae, or higher Protista. The terms “microbial cells” and “microbes” are used interchangeably with the term microorganisms.

The term “prokaryotes” is art recognized and refers to cells which contain no nucleus or other cell organelles. The prokaryotes are generally classified in one of two domains, the Bacteria and the Archaea.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of rRNA analysis, the Archaea consist of four phylogenetically distinct groups: TACK (Thaumarchaeota, Aigarchaeota, Crenarchaeota and Korarchaeota), Asgaard, DPANN (Diapherotrites, Parvarchaeota, Aenigmarchaeota, Nanoarchaeota and Nanohaloarchaeota) and Euryarchaeota. On the basis of their physiology, the Archaea can be organized into at least three types: methanogens (prokaryotes that produce methane); extreme halophiles (prokaryotes that live at very high concentrations of salt (NaCl); and extreme (hyper) thermophiles (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats.

“Bacteria”, or “Eubacteria”, refers to a domain of prokaryotic organisms. Now Bacteria include about 80 distinct bacterial phyla with numbers steadily growing. They were originally grouped into only 11 divisions by Woese and other scientists at the time (1987) as follows: (1) Gram-positive (gram+) bacteria, of which there are two major subdivisions, (a) high G+C group (Actinomycetes, Mycobacteria, Micrococcus, others) and (b) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include, for example, cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Stenotrophomonas, Spirilla, Serratia, Vibrio, Rhizobium, Chlamydia, Rickettsia, Treponema, and Fusobacterium.

“Gram positive bacteria” include, for example, cocci, nonsporulating rods, and sporulating rods. The genera of Gram positive bacteria include, for example, Actinomyces, Bacillus, Clostridium, Corynebacterium, Erysipelothrix, Kocuria, Lactobacillus, Listeria, Mycobacterium, Myxococcus, Nocardia, Paenibacillus, Staphylococcus, Streptococcus, and Streptomyces.

Preferably, in the microbial Cytometric Mock Community of the present invention the at least three different microbial species comprise or consist of species derived from archaea, bacteria, fungi, protozoa and algae, preferably derived from bacterial, fungal and/or algae species, more preferably from bacterial species. More preferably, in the microbial Cytometric Mock Community according to the invention the three different microbial species are selected from Kocuria rhizophila, Paenibacillus polymyxa, Stenotrophomonas rhizophila and Escherichia coli, even more preferably from the strains Kocuria rhizophila DSM 348, Paenibacillus polymyxa DSM 36, Stenotrophomonas rhizophila DSM 14405 and Escherichia coli DSM4230. In a particular preferred embodiment, the at least three different microbial species of the microbial Cytometric Mock Community of the invention are the strains Kocuria rhizophila DSM 348, Stenotrophomonas rhizophila DSM 14405 and at least one of Paenibacillus polymyxa DSM 36 and Escherichia coli DSM4230.

It is known in the art that cells of certain microorganisms differ in average size and/or average cell volume depending on the state the cells are in. In order to arrive at a microbial Cytometric Mock Community according to the invention, wherein variations in average cell size and/or average cell volume of a given microbial species are minimized, it is advantageous to use in the microbial Cytometric Mock Community of the invention microbial cells that are derived from cultures that are in stationary state. In other words, it is preferable that the cells of the at least three different microbial species of the microbial Cytometric Mock Community of the invention are derived from separate cultures each being in stationary state.

In the microbial Cytometric Mock Community of the present invention, the at least three different microbial species are selected such that, when measured using flow cytometry, the specific gate pattern of each microbial species differs significantly from the specific gate patterns of the other microbial species of the microbial Cytometric Mock Community of the invention. The specific gate patterns of two microbial species differ significantly from each other if the overlap between the two species in a scatter plot is less than a pre-defined threshold value. The degree of accepted overlap, however, can be defined prior to each new experiment. In a preferred embodiment, the threshold value is 10% or less. In other words, if not more than 10% of the cells of the first microbial species are located in a gate assigned to the second microbial species and the other way round, the gate patterns of the two microbial species are held to differ significantly.

In order to ensure a significant difference in the specific gate pattern between the at least three different microbial species of the microbial Cytometric Mock Community of the invention, the species are selected based on physico-chemical properties of the cells of the respective species. Preferably, the at least three different microbial species of the microbial Cytometric Mock Communities of the invention differ in one or more of:
relative DNA content,
relative genomic content of the nucleobases guanine (G) and cytosine (C) (e.g. in % of genomic G and C content; also referred to as G+C content),
relative cell size,
Gram +/− affiliation, and/or
capacity to form spores.

As used herein, the term “relative DNA content” is used in its art recognized meaning and refers to the different fluorescence intensities of individual cells after staining with a DNA specific fluorescent dye.

Relative DNA content can be determined using fluorescence staining of the DNA with a specific fluorescent dye. Preferably, the dye DAPI (4′,6-diamidino-2-phenylindole) can be used which very specifically stains A+T rich regions of the DNA of a cell. However, also any other DNA or highly resolving nucleic acid dye can be used determining relative DNA content.

As used herein, relative genomic GC-content (or guanine-cytosine content) is the percentage of the nitrogenous bases guanine or cytosine from the four different bases, also including adenine and thymine, in total genomic DNA of an organism (whole genome). GC content is usually expressed as a percentage value, but sometimes as a ratio (called G+C ratio or GC-ratio). GC-content percentage is calculated as:

[(G+C):(G+C+A+T)]×100%

whereas the G+C-ratio is calculated as: (G+C):(A+T)

The GC-content percentages as well as G+C-ratio can be measured by several means which are well known to the person skilled in the art, preferably if the genome of the respective microbial species or strain has been sequenced then the GC-content or GC-ratio can be calculated by simple arithmetic. Alternatively, GC-content is measured using the melting temperature of the DNA double helix using spectrophotometry. The absorbance of DNA at a wavelength of 260 nm increases fairly sharply when the double-stranded DNA separates into two single strands when sufficiently heated.

As used herein, relative cell size refers to the measurement of the forward scatter light that is obtained per individual cell by using a light source to measure refraction and diffraction on cell surfaces. Typically, relative cell size is measured using flow cytometry. In the microbial Cytometric Mock Community of the invention, the at least three different microbial species are used in a pre-defined ratio relative to each other. Preferably, the relative abundance of cells of the microbial species of the microbial Cytometric Mock Community is selected such that a gate pattern is achieved using flow cytometry, which is not dominated by gates of only one or two microorganisms but wherein gates of all different microorganisms of the microbial Cytometric Mock Community of the invention contribute in order to arrive at a well-balanced coverage of the dotplot with gates of the at least three different microorganisms. By doing so, it is ensured that the microbial Cytometric Mock Community of the inventions allows for preparation of a gate template which provides a well-balanced coverage of the part of the scatter plot which is of interest for further measurement using flow cytometry. Cells of the at least three microorganisms may be used in equal amounts or in any other relative combination that appears suitable for the purpose of the present invention. The exact ratio may depend on the choice of microbial species present in the microbial Cytometric Mock Community of the invention and on the purpose for which said microbial Cytometric Mock Community is intended. The person skilled in the art is well aware of techniques suitable to establish and define a desired ratio without undue burden.

Preferably, the at least three microorganisms of microbial Cytometric Mock Community of the invention comprise or consist of the three different microbial species are Kocuria rhizophila DSM 348, Stenotrophomonas rhizophila DSM 14405 and Paenibacillus polymyxa DSM 36 in a ratio of 19:1:80 e.g. if the cells have been grown on solid medium or 8:1:28 e.g. if cells have been grown in liquid medium.

In a further preferred embodiment, the at least three microorganisms of the microbial Cytometric Mock Community of the invention comprise or consist of cells of four different microbial species, wherein said species are the strains Kocuria rhizophila DSM 348, Stenotrophomonas rhizophila DSM 14405, Paenibacillus polymyxa DSM 36 and Escherichia coli DSM 4230, preferably in a ratio of 8:1:28:3 e.g. if cells have been grown in liquid medium.

In the microbial Cytometric Mock Community of the present invention, preferably cells of at least one, more than one or all different species of microorganisms present have been fixated. Fixation of the cells in the microbial Cytometric Mock Community of the invention ensures that unwanted change or modification of the cells are minimized or even avoided. By doing so, the shelf life of the microbial Cytometric Mock Community is improved and variation between different time points of use of the microbial Cytometric Mock Community or between different batches of the microbial Cytometric Mock Community of the invention is reduced. The person skilled in the art is well aware of different techniques for fixation of the microbial cells of the microbial Cytometric Mock Community of the invention. Exemplary embodiments of such techniques comprise fixation using para-formaldehyde (PFA), preferably a mixture of 1% to 4% PFA in PBS in combination with 70% ethanol in bi-distilled water and storage at −20° C.

In the microbial Cytometric Mock Community of the present invention, preferably cells of at least one, more than one or all different species of microorganisms present have been stained. The cells can be stained with one or more specific or non-specific agents. The person skilled in the art is well aware of different agents and techniques for staining of the microbial cells of the microbial Cytometric Mock Community of the invention. Preferably, the cells of the microbial Cytometric Mock Community of the invention are stained with a non-cell specific agent, a so called “all-cell” stain. Exemplary embodiments of such an all-cell stain are agents which bind to a cellular compound or ingredient which is present in virtually all cells like e.g. DNA. Preferred all-cell staining agents are DNA specific stains like e.g. DAPI (4′-6-diamidino 2-phenylindole) which binds to A-T rich regions of the minor groove of DNA, and cyanine dyes that bind to nucleic acids such as e.g. SYBR Green I (N′,N′-dimethyl-N-[4-[(E)-(3-methyl-1,3-benzothiazol-2-ylidene)methyl]-1-phenylquinolin-1-ium-2-yl]-N-propylpropane-1,3-diamine) as well as SYBR Safe ((Z)-4-((3-Methylbenzo[d]thiazol-2(3H)-ylidene)methyl)-1-propylquinolin-1-ium 4-methylbenzenesulfonate) or POPO™-3 Iodid (Benzoxazolium, 2,2′-[1,3-propanediylbis[(dimethyliminio)-3,1-propanediyl-1(4H)-pyridinyl-4-ylidene-1-propen-1-yl-3-ylidene]]bis[3-methyl]-, tetraiodide).

The microbial Cytometric Mock Community of the present invention may further comprise one or more types of beads with a given fluorescence intensity suitable for measurement in flow cytometry. By combining the use of different microorganisms of the microbial Cytometric Mock Community with the known technique of using beads detectable in flow cytometry, the resulting microbial Cytometric Mock Community combines advantages of both approaches. While the use of at least three different microorganisms allows for verification and calibration of the process and protocols of sample extraction, preparation and measurement, the use of fluorescent beads allows the addition to the gate template of the microbial Cytometric Mock Community of gates of well-defined size, intensity and remarkable reproducibility. Suitable types of beads with a given size and fluorescence intensity are well-known to the person skilled in the art. Such beads are commonly made of latex, polystyrol or the like. Preferably, the beads are chosen such that the beads exhibit a relative size which leads to bead gates in the gate template of the microbial Cytometric Mock Community of the invention that do not overlap with gates of the at least three microorganisms. In a preferred embodiment, the size of the beads is selected such to cover areas of the dotplot of the gate template of the microbial Cytometric Mock Community of the invention wherein no or only minor gates of the at least three microorganisms are present. Typically, the beads are selected to have an average bead size (diameter) selected from about 0.2 μm to 5 μm, preferably from about 0.5 μm to 3 μm. In general, the beads are selected to have a relative size which is in the range of the size of the microorganisms to be measured and, preferably, does not deviate by orders of magnitude from the relative size of all types of microorganisms of the microbial Cytometric Mock Community.

Furthermore, the present invention is directed to a method of generating a gate template for standardization of flow cytometric analysis, the method comprising the steps of:
providing a microbial Cytometric Mock Community of the invention;
fixating the microbial cells of the microbial Cytometric Mock Community;
staining the microbial cells of the microbial Cytometric Mock Community, preferably stained using an all-cell stain suitable for cytometric analysis, more preferably using a stain which binds to the DNA, even more preferably using DAPI or SYBR Green;
subjecting the stained microbial cells of the microbial Cytometric Mock Community to flow cytometric measurement; and
defining gates for the different microbial species of the microbial Cytometric Mock Community to form a gate template of the microbial Cytometric Mock Community.

In addition, the present invention is also directed to a method of analysing a sample by standardized flow cytometry, the method comprising the steps of:
providing a sample comprising microorganisms to be analysed by flow cytometry and a microbial Cytometric Mock Community of the invention;
processing the sample and the microbial Cytometric Mock Community in the same way, wherein processing encompasses fixation and staining of microbial cells, preferably staining using an all-cell stain suitable for cytometric analysis, more preferably using a stain which binds to the DNA, even more preferably using DAPI or SYBR Green;
subjecting the processed sample and processed microbial Cytometric Mock Community to flow cytometric measurement;
defining a gate template for standardisation by using the measurement data of the different microbial species of the microbial Cytometric Mock Community; and
analysing the measurement data acquired for the sample in relation to the gate template defined for the microbial Cytometric Mock Community.

The present invention is also directed to a kit comprising a microbial Cytometric Mock Community of the invention and a manual for performing one of the methods of the present invention.

Furthermore, the present invention is also directed to a use of a microbial Cytometric Mock Community of the invention in standardisation of flow cytometric measurement.

In the following, the present invention is further described by way of examples.

### FIGURES

FIG. 1: Creation of the gates for the microbial Cytometric Mock Communities. Left: Master gates (large geometric shape) which comprises 200,000 cells per measurement with gate templates (smaller gates). Two types of beads were included into each measurement (bead gates). Upper left: Master gate, gate template, and bead gates for the microbial Cytometric Mock Community cultivated on agar plates for 72 h. Lower left: Master gate, gate template, and bead gates for the microbial Cytometric Mock Community cultivated in liquid medium for 24 h. Right: Analyzed microbial Cytometric Mock Communities. Upper right: strains were cultivated on agar plates for 72 h, sampled, fixated, stained, and mixed at proportions 19:1:80 and measured cytometrically: Kocuria rhizophila DSM 348 (gates P4, P5, P6), Stenotrophomonas rhizophila DSM 14405 (gates P1, P2, P3), Paenibacillus polymyxa DSM 36 (gates P7, P8, P9, P10, P11). Lower right, strains that were cultivated in liquid medium for 24 h sampled, fixated, stained, and mixed at proportions 8:1:28:3 and measured cytometrically: Kocuria rhizophila DSM 348 (gates L5, L6, L7), Stenotrophomonas rhizophila DSM 14405 (gates L1, L2), Paenibacillus polymyxa DSM 36 (gates L8, L9, L10, L11, L12), and Escherichia coli DSM 4230 (gates L3, L4).

FIG. 2: Flow cytometric patterns of the cells that were cultivated on agar plates for 72 h. Left: Paenibacillus polymyxa DSM 36 (gates P7, P8, P9, P10, P11). Middle left: Stenotrophomonas rhizophila DSM 14405 (gates P1, P2, P3). Middle right: Kocuria rhizophila DSM 348 (gates P4, P5, P6). Right: microbial Cytometric Mock Community ‘Agar plate’. Per pure culture 50,000 cells and per master gate 200,000 cells were measured. Two types of beads were included in each measurement. Cell gates were set according to the gate template in FIG. 1.

FIG. 3: Flow cytometric patterns of the cells that were cultivated in liquid medium for 24 h. Left: Paenibacillus polymyxa DSM 36 (gates L8, L9, L10, L11, L12). Middle left: Stenotrophomonas rhizophila DSM 14405 (gates L1, L2). Middle: Kocuria rhizophila DSM 348 (gates L5, L6, L7). Middle right: Escherichia coli DSM 4230 (gates L3, L4). Right: microbial Cytometric Mock Community ‘Liquid medium’. Per pure culture 50,000 cells and per master gate 200,000 cells were measured. Two types of beads were included in each measurement. Cell gates were set according to the gate template in FIG. 1.

FIG. 4: Flow cytometric patterns of the cells that were cultivated over time in liquid medium for 24 h. Samples were taken at 0 h, and after 2 h, 4 h, and 24 hours of cultivation. First row: Paenibacillus polymyxa DSM 36 (gates L8, L9, L10, L11, L12). Second row: Stenotrophomonas rhizophila DSM 14405 (gates L1, L2). Third row: Kocuria rhizophila DSM 348 (gates L5, L6, L7). Forth row: Escherichia coli DSM 4230 (gates L3, L4). Per master gate 50,000 cells were measured. Two types of beads were included in each measurement. Cell gates were set according to the gate template in FIG. 1.

FIG. 5: NMDS-plot for determination of dissimilarity of technical and biological replicates of two microbial Cytometric Mock Communities originating from an agar plate and liquid medium. Euclidian distance calculation was used. The technical replicates showed the highest similarity while the two microbial Cytometric Mock Communities were the most dissimilar ones. Left community) (open circle): microbial Cytometric Mock Community ‘Agar plate’; right communities: 2 (triangle up): three biological replicates of the cytometric Cytometric Mock Community ‘Liquid medium’ and measured after 3 days storage at −20° C., 3 (straight cross): three biological replicates of the microbial Cytometric Mock Community ‘Liquid medium’ and measured after 2 months storage at −20 ° C., 4 (cross): technical replicates (of biological replicate nr. 2) of the microbial Cytometric Mock Community ‘Liquid medium’ measured after 2 months storage at −20 ° C., 5 (rhombus): technical replicates (of biological replicate nr. 3) of the microbial Cytometric Mock Community ‘Liquid medium’ measured after 2 months storage at −20 ° C., 6 (triangle down): technical replicates (of biological replicate nr. 1) of the microbial Cytometric Mock Community ‘Liquid medium’ measured after 2 months storage at −20 ° C., 7 (rectangle with cross): first repetitive measurement of 6 and 8, (star): second repetitive measurement of 6.

FIG. 6: Microbial Cytometric Mock Community ‘Liquid medium’ measured with a 355 nm laser at a constant laser power of 100 mW and with a 488 nm laser with decreasing laser power: A: 400 mW, B: 200 mW, C: 100 mW, D: 50 mW. The dotplot in E represents the microbial Cytometric Mock Community measured at 50 mW at an increased gain value for the FSC-PMT.

FIG. 7: Microbial Cytometric Mock Communities ‘Liquid medium’ analysed with DAPI that were created out of different proportions of the four strains from liquid culture, cultivated in liquid medium for 24 h. The strains were separately fixed, stained with DAPI and mixed in different proportions and measured. Per master gate 200,000 cells were measured. The proportions are given for Paenibacillus polymyxa DSM 36 (gates L8, L9, L10, L11, L12), Stenotrophomonas rhizophila DSM 14405 (gates L1, L2), Kocuria rhizophila DSM 348 (gates L5, L6, L7), Escherichia coli DSM 4230 (gates L3, L4), respectively. A) 70:2.5:20:7.5; B) 70:2.5:12.5:15; C) 81.4:2.8:14.4:1.4; D) 97.5:0.5:1.5:0.5; E) 45:5:15:35; F) 45:0.5:15:39.5.

FIG. 8: Microbial Cytometric Mock Communities ‘Agar plate’ analysed with DAPI that were created out of different proportions of the three strains from agar plate culture, cultivated for 72 h. The strains were separately fixed, stained with DAPI and mixed in different proportions and measured. Per master gate 200,000 cells were measured. The proportion are given for Paenibacillus polymyxa DSM 36 (gates P7, P8, P9, P10, P11) Stenotrophomonas rhizophila DSM 14405 (gates P1, P2, P3), Kocuria rhizophila DSM 348 (gates P4, P5, P6), respectively. A) 80:1:19; B) 80:19:1; C) 80:15:5; D) 53.3:42.7:4; E) 92:0.25:7.75; F) 60:10:30.

FIG. 9: Microbial Cytometric Mock Community ‘Agar plate’ analysed with SYBR Green that was created of the three strains from agar plate culture, cultivated for 72 h. The strains were separately fixed, stained with SYBR Green and mixed in the proportion of 33:33:33 and measured. Per pure culture 50,000 cells and per master gate 200,000 cells were measured. Left: Paenibacillus polymyxa DSM 36. Middle left: Stenotrophomonas rhizophila DSM 14405. Middle right: Kocuria rhizophila DSM 348. Right: microbial Cytometric Mock Community ‘Agar plate’. Two types of beads were included in each measurement.

FIG. 10: Microbial Cytometric Mock Community ‘Liquid medium’ analysed with SYBR Green that was created of the four strains from liquid medium culture, cultivated for 24 h. The strains were separately fixed, stained with SYBR Green and mixed in the proportion of 25:25:25:25 and measured. Per pure culture 50,000 cells and per master gate 200,000 cells were measured. Left: Paenibacillus polymyxa DSM 36. Middle left: Stenotrophomonas rhizophila DSM 14405. Middle: Kocuria rhizophila DSM 348. Middle right: Escherichia coli DSM 4230. Right: Cytometric Mock Community ‘Liquid medium’. Two types of beads were included in each measurement.

FIG. 11: Microbial Cytometric Mock Communities ‘Agar plate’ analysed with SYBR Green that were created out of different proportions of the three strains from agar plate culture, cultivated for 72 h. The strains were separately fixed, stained with SYBR Green and mixed in different proportions and measured. Per master gate 200,000 cells were measured. The proportions are given for Paenibacillus polymyxa DSM 36, Stenotrophomonas rhizophila DSM 14405, Kocuria rhizophila DSM 348. A) 55:31:14; B) 75:20:5; C) 40:50:10; D) 60:32.5:7.5; E) 60:37.5:2.5.

FIG. 12: Microbial Cytometric Mock Communities ‘Liquid medium’ analysed with SYBR Green that were created out of different proportions of the four strains from liquid culture, cultivated in liquid medium for 24 h. The strains were separately fixed, stained with SYBR Green and mixed in different proportions and measured. Per master gate 200,000 cells were measured. The proportions are given for Paenibacillus polymyxa DSM 36, Stenotrophomonas rhizophila DSM 14405, Kocuria rhizophila DSM 348, Escherichia coli DSM 4230. A) 50:15:15:20; B) 75:11:5:9; C) 40:12.5:12.5:35; D) 64.5:7.5:18:10; E) 75:2.5:10.5:12.

### EXAMPLES

### Results

### Cultivation of Strains for the Microbial Cytometric Mock Community

The four strains Kocuria rhizophila DSM 348, Paenibacillus polymyxa DSM 36, Stenotrophomonas rhizophila DSM 14405, and Escherichia coli DSM 4230 were chosen to construct two different microbial Cytometric Mock Communities. For the first microbial Cytometric Mock Community, the strains were independently cultivated on LB agar plates for 72 hours. For the second microbial Cytometric Mock Community the respective strains were independently cultivated in liquid LB medium by taking one colony from agar plates (after 72 h) and its pre-cultivation for 24 h in liquid medium. The main cultures were started by inoculation of 1 ml (OD700 nm d=0.05) of the pre-culture and grown for another 24 h (cf. methods). The final four stationary state cultures served to create the microbial Cytometric Mock Community from liquid medium.

### Creation of the Gate Templates

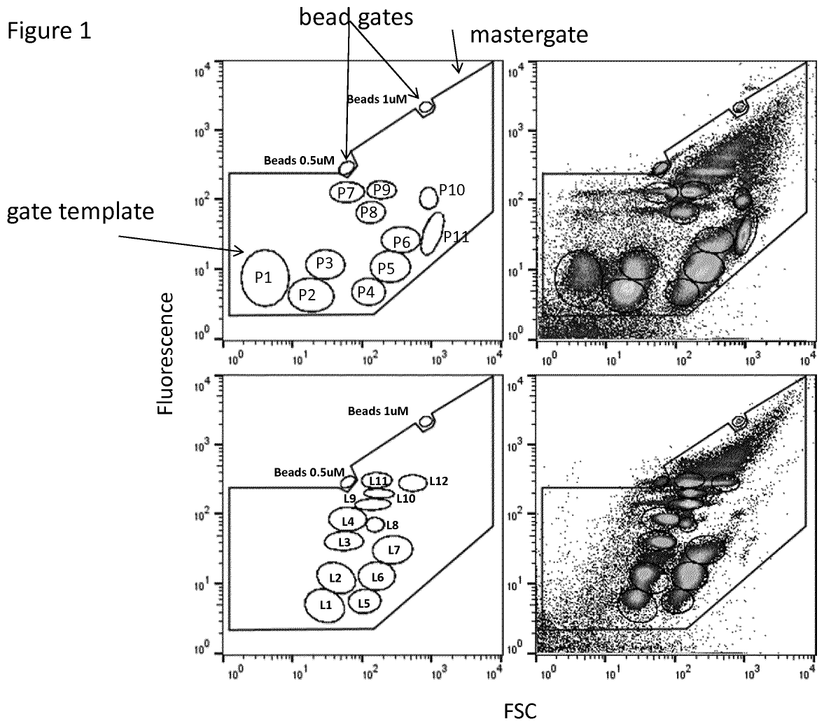

Stationary state liquid cultures and agar plate cultures were used to ensure stable populations states which are represented by discrete cytometric population patterns. These patterns are not homogeneous. DAPI/FSC pattern of a population describe cell size related cell characteristics and numbers of chromosomes per cell. A bacterial cell usually has one, sometimes also two or three chromosomes of different sizes and sequences. In addition, depending on states in the growth cycle bacteria can have many copies of a chromosome. The relative numbers of chromosomes per cell can be detected by DAPI staining. Therefore, a DAPI/FSC dotplot mirrors the heterogeneity of a population with regard to cell size and chromosome number of cells that cluster in different subpopulations. The position of cell clusters in a histogram and the numbers of clusters are strain specific and depend frequently on growth stages. All upcoming subpopulations can be marked by gates (FIG. 1). This fact is used to create strain specific gate templates within a master gate that comprises all gates (FIG. 1). Spiked beads of different types serve as control for the stable position of the master gate and the gate templates (FIG. 1, two gates outside the master gate).

To create a gate template for a microbial Cytometric Mock Community it is advisable to choose cells from relatively stable growth stages. For our two microbial Cytometric Mock Communities we used, as a first growth stage, the 72 h grown agar plate cultures and, as a second growth stage, the 24 h grown stationary state states liquid cultures. The following numbers of gates were defined for the four strains and the two growth stages, respectively: Kocuria rhizophila DSM 348 (3,3), Paenibacillus polymyxa DSM 36 (5,5), Stenotrophomonas rhizophila DSM 14405 (3,2), and Escherichia coli DSM 4230 (0,2). Gates of Escherichia coli DSM 4230 were found to overlap with gates from Paenibacillus polymyxa DSM 36 when cultivated on agar plates, therefore, we excluded this strain from the agar plate microbial Cytometric Mock Community.

### Cytometric Patterns of Cells From Agar Plates

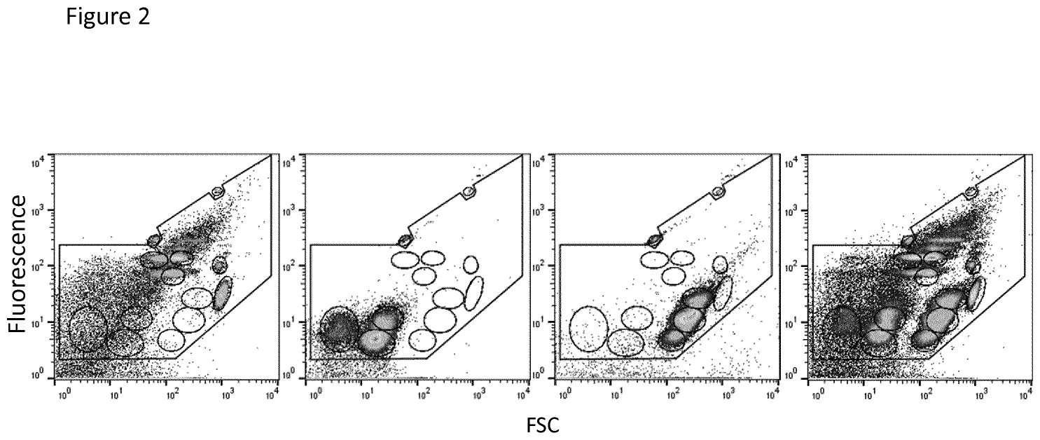

Cells of each strain were harvested from LB agar plates after 72 h, fixated and stained with DAPI and measured by flow cytometry. The strain specific patterns are shown in FIG. 2. The microbial Cytometric Mock Community ‘Agar Plate’ is created by mixing cells at proportions 19:1:80 and measuring them cytometrically: Kocuria rhizophila DSM 348 (gates P4, P5, P6), Stenotrophomonas rhizophila DSM 14405 (gates P1, P2, P3), and Paenibacillus polymyxa DSM 36 (gates P7, P8, P9, P10, P11).

Following the mentioned protocol, the agar plate cultures produce cell material for as much as 100 calibrations. The fixated cells must be stored at −20° C.

### Cytometric Patterns of Cells From Liquid Media

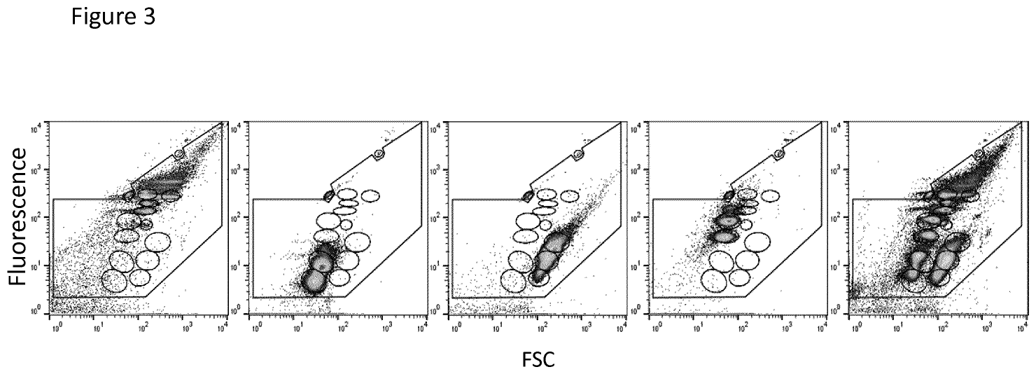

Cells of each strain were harvested from 24 h grown stationary state liquid cultures, fixated and stained with DAPI and measured by flow cytometry. The specific patterns are shown in FIG. 3. The microbial Cytometric Mock Community ‘Liquid Medium’ is created by mixing cells at proportions 8:1:28:3 and measured cytometrically: Kocuria rhizophila DSM 348 (gates L5, L6, L7), Paenibacillus polymyxa DSM 36 (gates L8, L9, L10, L11, L12), Stenotrophomonas rhizophila DSM 14405 (gates L1, L2), and Escherichia coli DSM 4230 (gates L3, L4).

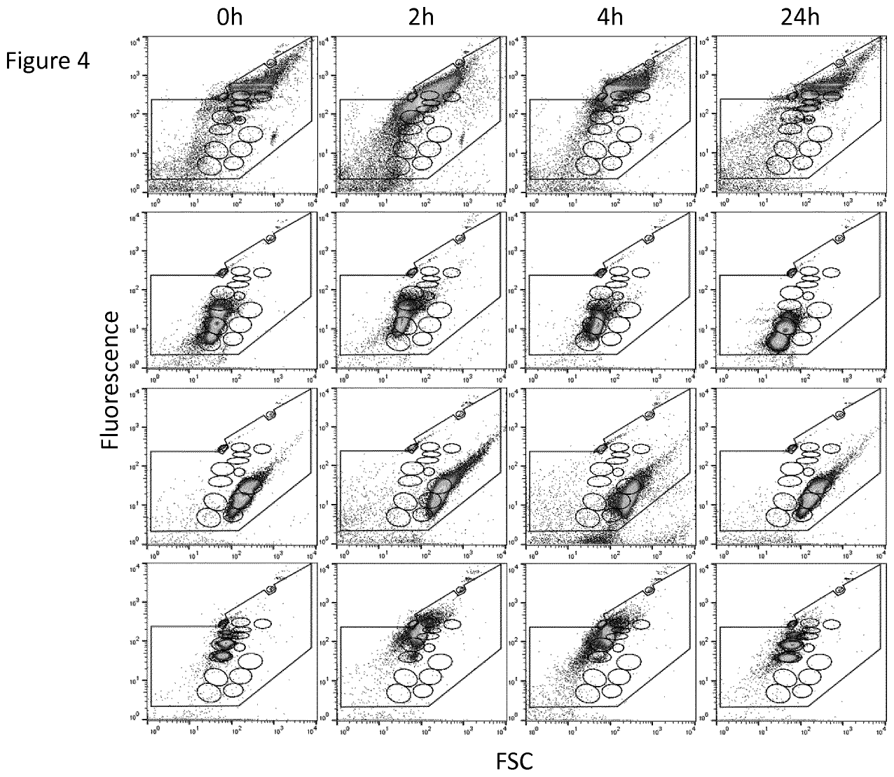

We also followed the different growth stages of the four strains in liquid culture after 0 h, 2 h, 4 h, and 24 h (FIG. 4). The cell cycle of a pure strain, i.e. increase in chromosome copy numbers per cell due to DNA replication or decrease due to cell division, can be observed by the distribution of cells within strain specific gates which are in this study the gates L8, L9, L10, L11, L12 for Paenibacillus polymyxa DSM 36, the gates L1, L2 for Stenotrophomonas rhizophila DSM 14405, the gates L5, L6, L7 for Kocuria rhizophila DSM 348, and the gates L3, L4 for Escherichia coli DSM 4230. The four strains were grown independently on liquid LB medium as biological triplicates and were inoculated with stationary state cultures, respectively. All strains reached the stationary state after 24 h where the flow cytometric population pattern of the inoculum at 0 h was identical to the pattern reached after 24 h. During exponential growth the cells did contain generally more DNA and did not cluster to clearly separated subpopulations.

Following the mentioned protocol, one batch of cultures produces cell material for as much as 100 calibrations. The fixated cells must be stored at −20° C.

### Intrinsic Variation of Biological and Technical Samples in Flow Cytometric Patterns

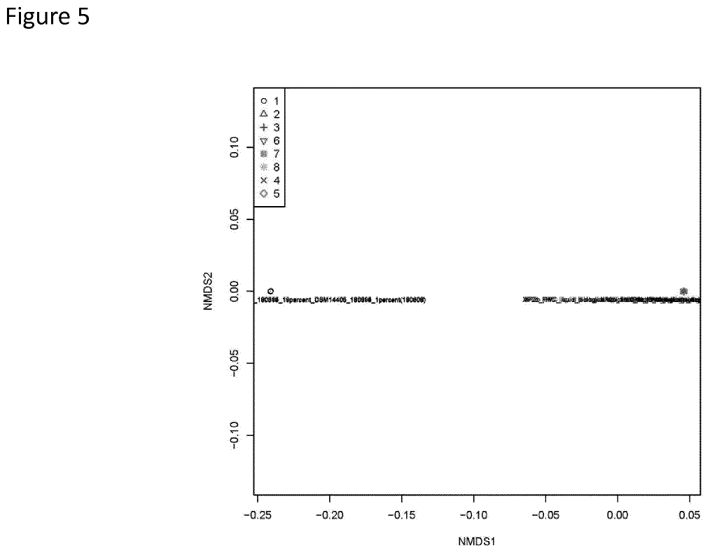

To ensure the quality and the reliability of the data, biological as well as technical replicates of the two microbial Cytometric mock Communities from the agar plates and from the liquid culture were generated and cytometrically measured. The two respective main gate templates (see above) were used to evaluate the triplicate measurements by using the flowCHIC (https://www.bioconductor.org/packages/release/bioc/html/flowCHIC.html). The degree of deviation between the cytometrically measured biological and technical replicates was determined. The deviations between all technical samples showed extremely low Euclidian distance values. In contrast, the deviation between the microbial Cytometric Mock Community from the ‘agar plates’ and all samples from the ‘liquid medium’ was high according the Euclidian distance values (FIG. 5).

### Influence of the Laser Power on the Flow Cytometric Fingerprints

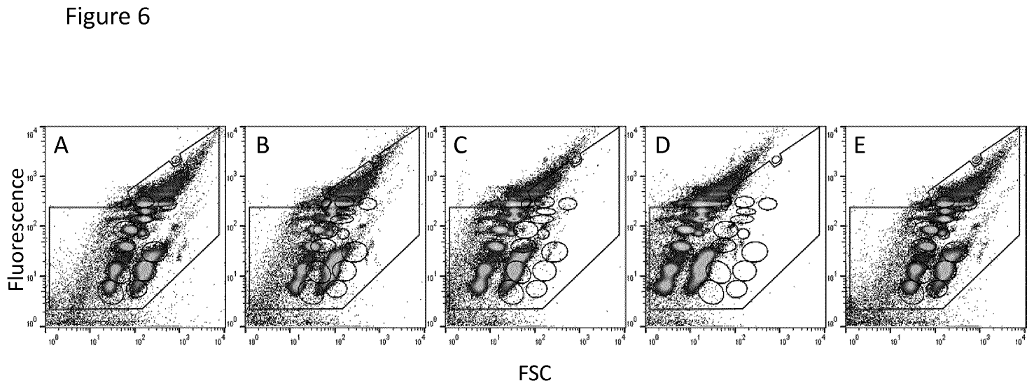

Flow cytometers are not only equipped with different laser types and wavelengths, the power of the lasers can also be different. Increasing laser power certainly influences the fluorescence intensity values of a cell by creating higher photon numbers. Low-cost flow cytometers are often equipped with low-cost low-power lasers, therefore, we wanted to test if low-power lasers resolve the scatter of microbial Cytometric Mock Community members accurately. While the 355 nm laser line of the Influx was a fixed line with a power of 100 mW, the 488 nm laser was equipped with an adjustable power option which was used to analyze the microbial Cytometric Mock Community of the liquid culture at 400 mW, 200 mW, 100 mW, and 50 mW (FIG. 6; A, B, C, D, respectively). A microbial Cytometric Mock Community was used that was stored at −20° C. We found the microbial Cytometric Mock Community to shift by about 1.2 magnitudes to lower values in the forward scatter channel in comparison to the gate template (FIG. 6). Although the distribution and resolution of the microbial Cytometric Mock Community members did not change it became clear that the laser power certainly influenced the position of the microbial Cytometric Mock Community in the histogram. We usually recommend placing the microbial Cytometric Mock Community into the middle of the histogram to enable the measurement of both smaller and larger cells in follow-up experiments. Therefore, we tested, if increasing the gain of the FSC-PMT could replace the microbial Cytometric Mock Community into its former position. This was possible, and thus, even low-power lasers guaranty a high resolution of the members of a microbial Cytometric Mock Community (FIG. 6 E).

### Influence of Different Proportions of Strains From Liquid Culture on Microbial Cytometric Mock Community Pattern Using DAPI

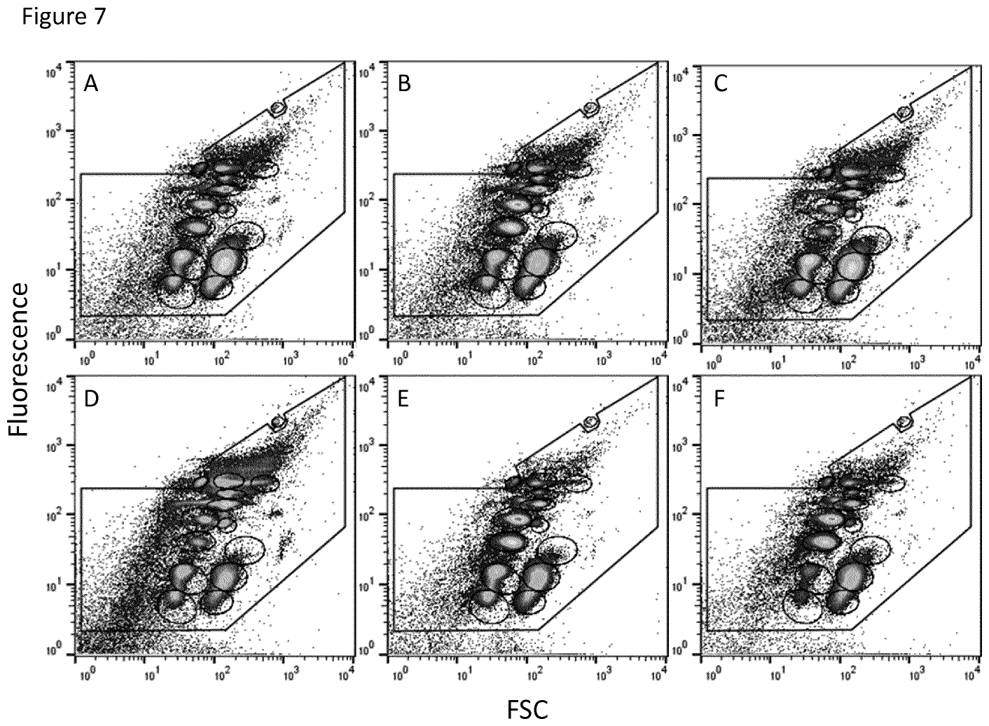

We analysed different proportions of strains within the microbial Cytometric Mock Community when stained with DAPI in order to test if other proportions might also be useful or might distort the structure of the microbial Cytometric Mock Community. All four strains were obtained from liquid cultures, respectively, and cultivated in liquid medium for 24 h. The strains were separately fixed, stained with DAPI and mixed in different proportions and measured. Per master gate 200,000 cells were measured. The proportions are given for Paenibacillus polymyxa DSM 36 (gates L8, L9, L10, L11, L12), Stenotrophomonas rhizophila DSM 14405 (gates L1, L2), Kocuria rhizophila DSM 348 (gates L5, L6, L7), Escherichia coli DSM 4230 (gates L3, L4), respectively. A) 70:2.5:20:7.5; B) 70:2.5:12.5:15; C) 81.4:2.8:14.4:1.4; D) 97.5:0.5:1.5:0.5; E) 45:5:15:35; F) 45:0.5:15:39.5. All proportions show well resolved microbial Cytometric Mock Community patterns and can all be used for cytometric calibration (FIG. 7).

### Influence of Different Proportions of Strains From Plate Culture on Microbial Cytometric Mock Community Pattern Using DAPI

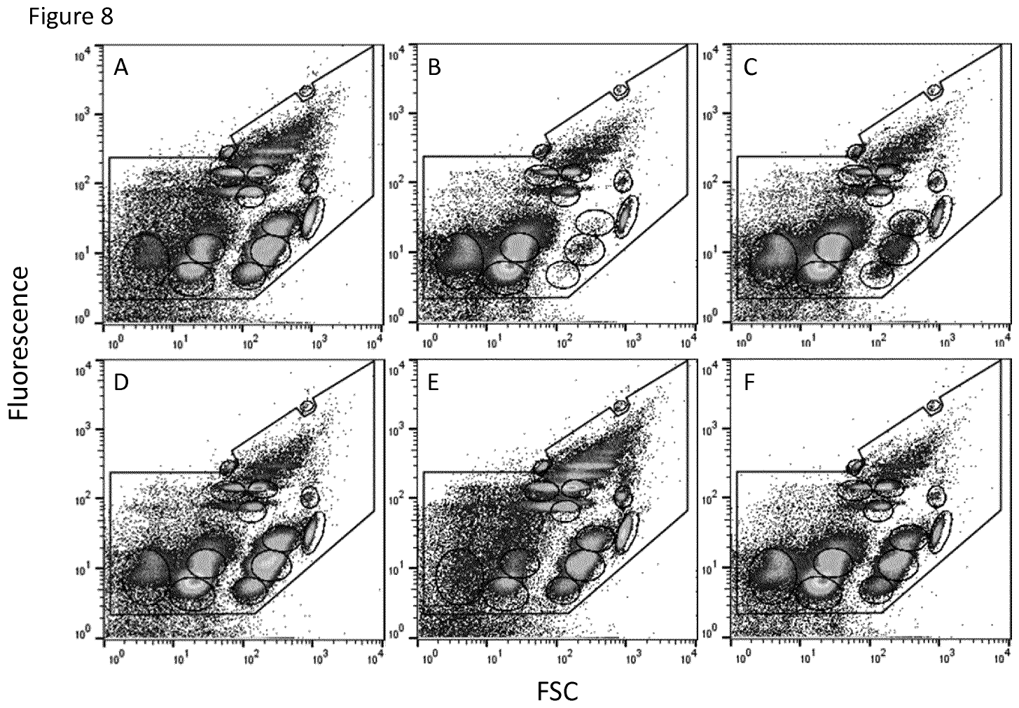

We analysed different proportions of strains within the microbial Cytometric Mock Community when stained with DAPI in order to test if other proportions might also be useful or might distort the structure of the microbial Cytometric Mock Community. All three strains were obtained from plate cultures, respectively, and cultivated on plates for 72 h. The strains were separately fixed, stained with DAPI and mixed in different proportions and measured. Per master gate 200,000 cells were measured. The proportions are given for Paenibacillus polymyxa DSM 36 (gates P7, P8, P9, P10, P11) Stenotrophomonas rhizophila DSM 14405 (gates P1, P2, P3), Kocuria rhizophila DSM 348 (gates P4, P5, P6), respectively. A) 80:1:19; B) 80:19:1; C) 80:15:5; D) 53.3:42.7:4; E) 92:0.25:7.75; F) 60:10:30 (FIG. 8).

### Influence of SYBR Green Staining on the Structure of the Microbial Cytometric Mock Community Pattern Originating From Plate Cultures

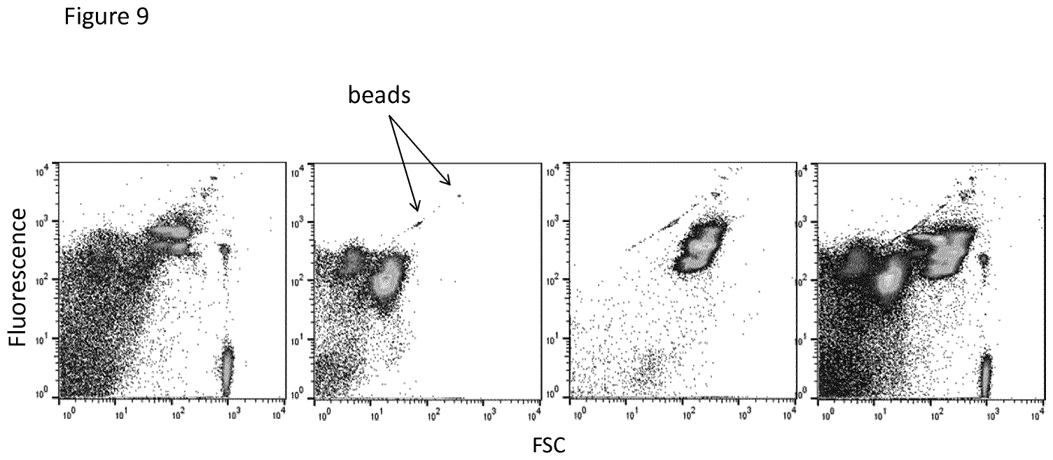

Many common flow cytometers are not equipped with an UV laser which is necessary to excite DAPI: Therefore, we tested the nucleic acid dye SYBR Green (excitation 488 nm) for its usefulness to stain the microbial Cytometric Mock Community and create well resolved patterns useful for calibration of such cytometers. We found that the patterns were not as highly resolved as was possible with DAPI, but nevertheless high enough to be useful as a microbial Cytometric Mock Community. Three strains, originating from agar plate culture, were cultivated for 72 h: Paenibacillus polymyxa DSM 36, Stenotrophomonas rhizophila DSM 14405, and Kocuria rhizophila DSM 348. The strains were separately fixed, stained with SYBR Green and mixed in the proportion of 33:33:33 and measured as ‘Agar plate’ microbial Cytometric Mock Community. Per pure culture 50,000 cells and per master gate 200,000 cells were measured and two types of beads were included in each measurement (FIG. 9).

### Influence of SYBR Green Staining on the Structure of the Microbial Cytometric Mock Community Pattern Originating From Liquid Cultures

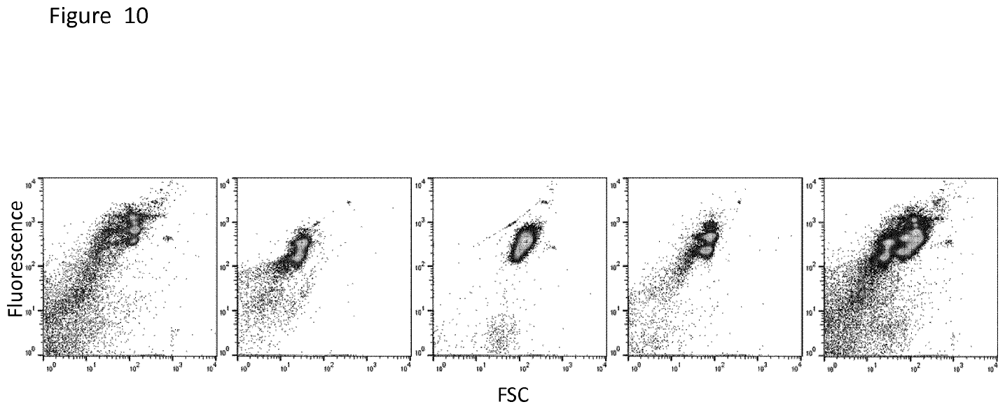

We also tested the nucleic acid dye SYBR Green (excitation 488 nm) for its usefulness to stain the microbial Cytometric Mock Community and create well resolved patterns useful for calibration of such cytometers from liquid cultures. We found again that the patterns were not as highly resolved as was possible with DAPI, but nevertheless high enough to be useful as a microbial microbial Cytometric Mock Community. Four strains, originating from agar plate culture, were cultivated for 24 h: Paenibacillus polymyxa DSM 36, Stenotrophomonas rhizophila ; DSM 14405, Escherichia coli DSM 4230 and Kocuria rhizophila DSM 348. The strains were separately fixed, mixed in the proportion of 25:25:25:25, stained with SYBR Green and measured as ‘Liquid medium’ microbial Cytometric Mock Community. Per pure culture 50,000 cells and per master gate 200,000 cells were measured and two types of beads were included in each measurement (FIG. 10).

### Influence of Different Proportions of Strains from Agar Plate Culture on Microbial Cytometric Mock Community Pattern Using SYBR Green

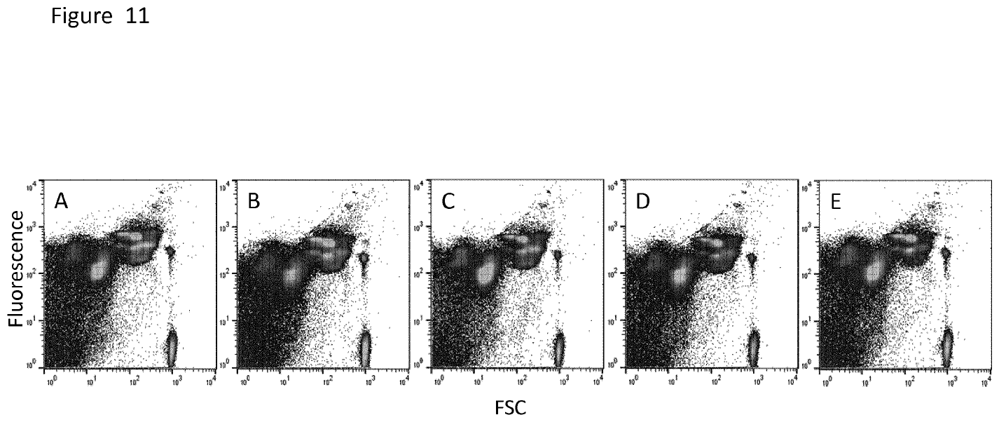

We also analysed different proportions of strains within the microbial Cytometric Mock Community when stained with SYBR Green in order to test if other proportions might also be useful or might distort the structure of the microbial Cytometric Mock Community. All three strains were obtained from agar plate cultures, respectively, and cultivated on agar plates for 72 h. The strains were separately fixed, mixed in different proportions, stained with SYBR Green and measured. Per master gate 200,000 cells were measured. The proportions are given for Paenibacillus polymyxa DSM 36, Stenotrophomonas rhizophila DSM 14405, Kocuria rhizophila DSM 348. A) 55:31:14; B) 75:20:5; C) 40:50:10; D) 60:32.5:7.5; E) 60:37.5:2.5. All proportions show well resolved microbial Cytometric Mock Community patterns (although not as well resolved as with DAPI) and can all be used for cytometric calibration (FIG. 11).

### Influence of Different Proportions of Strains from Liquid Culture on microbial Cytometric Mock Community Pattern Using SYBR Green

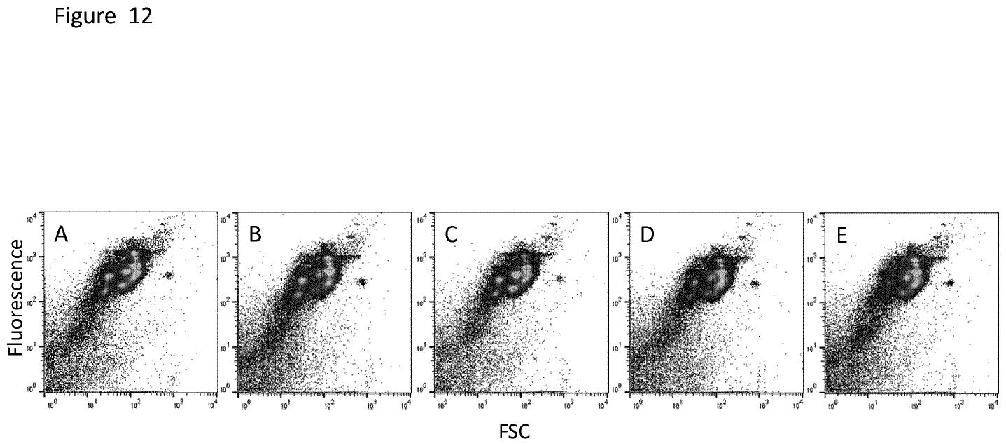

We analysed different proportions of strains within the microbial Cytometric Mock Community when stained with SYBR Green in order to test if other proportions might also be useful or might distort the structure of the microbial Cytometric Mock Community. All four strains were obtained from liquid cultures, respectively, and cultivated in liquid culture for 24 h. The strains were separately fixed, mixed in different proportions, stained with SYBR Green and measured. Per master gate 200,000 cells were measured. The proportions are given for Paenibacillus polymyxa DSM 36, Stenotrophomonas rhizophila DSM 14405, Kocuria rhizophila DSM 348, Escherichia coli DSM4230. A) 50:15:15:20; B) 75:11:5:9; C) 40:12.5:12.5:35; D) 64.5:7.5:18:10; E) 75:2.5:10.5:12. All proportions show well resolved microbial Cytometric Mock Community patterns (although not as well resolved as with DAPI) and can all be used for cytometric calibration (FIG. 12).

### Material and Methods

### Strains and Cultivation

The Cytometric Mock Community was constructed using following strains from the DSMZ: Kocuria rhizophila DSM 348, Paenibacillus polymyxa DSM 36, Stenotrophomonas rhizophila DSM 14405, Escherichia coli DSM 4230. The strains were handled following the DSMZ's recommendations, placed on LB-agar plates (Lysogeny Broth, Yeast extract 5 g/L, NaCl 5 g/L, Tryptone 10 g/L, pH 7.0, Agar 20 g/L, Carl ROTH GmbH, Karlsruhe, Germany) at 30° C. for 72 h. Afterwards a colony served as inoculum for a 100 mL liquid flask containing 20 mL of LB medium which was grown at 30° C. for 24 h at 150 rpm. This pre-cultivation step was done in triplicates and OD measured (d_{λ700nm}=0.5 cm, Ultrospec III Amersham Biosciences Europe). Following, triplicate 500 mL flasks, filled with 100 mL of LB medium, were inoculated to an OD=0.05 (d_{λ700}nm=0.5 cm) with cells of the pre-culture and grown at 30° C. for 24 h at 150 rpm.

### Cell Sampling and Fixation

Of the cell suspension 5 to 8 mL were taken, centrifuged (3.200 g, 10 min, 4° C.) and the supernatant discarded. The cells were washed in 3 ml phosphate buffered saline (PBS: 6 mM Na₂HPO₄, 1.8 mM NaH₂PO₄, 145 mM NaCl in bi-destilled H₂O, pH 7) once (3.200 g, 15 min, 4° C.) and the supernatant discarded. The cells were stabilized by adding 8 mL of para-formaldehyde solution (PFA, 2% in PBS) to the cell pellet for 30 min at room temperature. For a homogenized reaction, the pellet should be vortexed. After another centrifugation step (3.200 g, 15 min, 4° C., and discarding the supernatant), 8 mL of ethanol (70% in bi-distilled water) were added for fixation and the cell solution stored at −20° C. for two months maximum.

### Cell Staining: DAPI

The OD (d_{λ700nm}=0.5 cm) of the fixated cells was adjusted to 0.04 with PBS. Two ml of this solution were centrifuged (3.200 g, 15 min, 4° C.) and the supernatant discarded. The cell-pellet was resuspended in 1 mL of permeabilization buffer (0.1 M citric acid, 4.1 mM Tween 20, bi-destilled H₂O) and incubated for 20 min at room temperature After a further centrifugation step the supernatant was discarded and the cells were resuspended in 2 ml 0.24 μM DNA-DAPI staining solution (4′,6-di-amidino-2-phenyl-indole, Sigma-Aldrich, St. Louis, USA) in Na₂HPO₄/NaH₂PO₄ buffer (289 mM Na₂HPO₄, 128 mM NaH₂PO₄ with bi-distilled H₂O, pH 7) for subsequent staining overnight in the dark until flow cytometric measurement. Samples were filtered through 50 μm CellTrics® (Partec, Germany) prior to cytometric measurement. Fluorescence beads ((0.5 and 1 μm BB Fluoresbrite Microspheres (18339, 17458; Polysciences, Warrington, Pa., USA)) were added to the samples as internal standard. For measurements of single strains 50,000 and of microbial Cytometric Mock Communities 200,000 cells, respectively, were recorded.

### Cell Staining: SYBR Green

The preparation of the cells for the staining was identical to the method above. In short, the fixated cells were adjusted to an OD (d_{λ700nm}=0.5 cm) of 0.04 with PBS and 2 ml of this solution centrifuged (3.200 g, 10 min, 4° C.). The cells were pre-incubated for 4 min at 37° C., SYBR Green I (ThermoFisher Scientific, Waltham, Mass., USA) was added (final conc. 0.1×), and the cells were incubated at 37° C. for 20 min before measurement. Fluorescence beads (0.5 μm FluoSpheres carboxylate-modified microspheres, yellow-green fluorescent (505/515); F8813; and 1.0 μm FluoSpheres polystyrene microspheres, yellow-green fluorescent (505/515), F13081; ThermoFischer Sci.) were added to the samples as internal standard. For measurements of single strains 50,000 and of microbial Cytometric Mock Communities 200,000 cells, respectively, were recorded.

### Flow Cytometric Analysis

Cytometric measurements were performed with a BD Influx v7 Sorter USB, (Becton, Dickinson and Company, Franklin Lakes, USA) equipped with a blue 488 nm Sapphire OPS laser (400 mW) and a 355 nm Genesis OPS laser (100 mW, both Coherent, Santa Clara, Calif., USA).

The 488 nm laser was used for analysis of forward scatter (FSC, 488/10), side scatter (SSC, trigger signal, 488/10), and the SYBR Green I fluorescence (530/40), while the 355 nm laser excited the DAPI fluorescence (460/50). Light was detected by Hamamatsu R3896 PMTs in C6270 sockets (Hamamatsu, 211 Hamamatsu City, Japan). The fluidic system was run at 33 psi with sample overpressure at 0.5 psi and a 70 μm nozzle. The sheath fluid consisted of FACSFlow buffer (BD) sample. Samples were analyzed at a speed of 2500 events s⁻¹. Cytometric data were evaluated using FlowJo v10.0.8r1 with the Engine v3.04910 (FlowJo, LLC, Ashland, USA) and the R packages flowCyBar and flowCHIC (Bioconductor platform).

### CONCLUSION

If we want to bring flow cytometry to the next level and help to shape and develop micro-ecology, health (microbiome) and biotechnology fields during the upcoming years, standardization is one of the mandatory steps to proceed to new levels of knowledge as it will allow creating standardized and comparable data between studies and labs. We are certain that standardization will help ecologists, microbiologists, molecular biologists and flow cytometrists to exchange hypothesis and increase scientific knowledge by working together and comparing data on a standardized basis. We are certain that the Microbial Cytometric Mock community allows the measurement of accurate population or community dynamics in a much better way than it is possible to date and will help to analyze dynamics of microbial communities in many applications such as environment, human and animal health or in biotechnology.

1. Microbial Cytometric Mock Community for use in flow cytometric analysis, the microbial Cytometric Mock Community comprising or consisting of cells of at least three different microbial species in a pre-defined ratio, wherein the at least three different microbial species are selected such that, when measured using flow cytometry, the specific gate pattern of each microbial species differs significantly from the specific gate pattern of the other microbial species of the microbial Cytometric Mock Community, preferably the at least three different microbial species differ in overall DNA content, relative genomic GC-content, average cell size, Gram +/− affiliation and/or capacity to form spores.

2. Microbial Cytometric Mock Community of claim 1, wherein the at least three different microbial species comprise or consist of species derived from archaea, bacteria, fungi, protozoa and algae, preferably derived from bacterial species.

3. Microbial Cytometric Mock Community according to claim 1, wherein the cells of the at least three different microbial species are derived from cultures each being in stationary state.

4. Microbial Cytometric Mock Community according to claim 1, wherein the cells of the at least three different microbial species have been fixated and, optionally, stained with nucleic acid specific fluorescent dyes.

5. Microbial Cytometric Mock Community according to claim 1, wherein the three different microbial species are selected from Kocuria rhizophila, Paenibacillus polymyxa, Stenotrophomonas rhizophila and Eschericha coli, preferably from the strains Kocuria rhizophila DSM 348, Paenibacillus polymyxa DSM 36, Stenotrophomonas rhizophila DSM 14405 and Eschericha coli DSM 4230.

6. Microbial Cytometric Mock Community according to claim 1, wherein the at least three different microbial species are Kocuria rhizophila DSM 348, Stenotrophomonas rhizophila DSM 14405 and at least one of Paenibacillus polymyxa DSM 36 and Eschericha coli DSM 4230.

7. Microbial Cytometric Mock Community according to claim 1, wherein the microorganisms of microbial Cytometric Mock Community comprise or consist of the three different microbial species are Kocuria rhizophila DSM 348, Stenotrophomonas rhizophila DSM 14405 and Paenibacillus polymyxa DSM 36.

8. Microbial Cytometric Mock Community according to claim 1, wherein the microorganisms of microbial Cytometric Mock Community comprise or consist of cells of four different microbial species, wherein said species are the strains Kocuria rhizophila DSM 348, Stenotrophomonas rhizophila DSM 14405, Paenibacillus polymyxa DSM 36 and Eschericha coli DSM 4230.

9. Microbial Cytometric Mock Community according to claim 1, wherein the microbial Cytometric Mock Community further comprises one or more types of beads suitable for flow cytometric measurement, preferably if more than one type of beads is present, the types of beads are selected such that their gates do not overlap with those of the cells when measured using flow cytometry.

10. A method of generating a gate template for standardization of flow cytometric analysis, the method comprising the steps of:
providing the microbial Cytometric Mock Community of claim 1;
fixating the microbial cells of the microbial Cytometric Mock Community;
staining the microbial cells of the microbial Cytometric Mock Community;
subjecting the stained microbial cells of the microbial Cytometric Mock Community to flow cytometric measurement; and
defining the gates for the different microbial species of the microbial Cytometric Mock Community to form a gate template of the microbial Cytometric Mock Community.

11. A method of analysing a sample by standardized flow cytometry, the method comprising the steps of:
providing a sample comprising microorganisms to be analysed by flow cytometry and the microbial Cytometric Mock Community of claim 1;
processing the sample and the microbial Cytometric Mock Community in the same way, wherein processing encompasses fixation and staining of microbial cells;
subjecting the processed sample and processed microbial Cytometric Mock Community to flow cytometric measurement;
defining a gate template for standardisation by using the measurement data of the different microbial species of the microbial Cytometric Mock Community; and
analysing the measurement data acquired for the sample in relation to the gate template defined for the microbial Cytometric Mock Community.

12. A kit comprising the microbial Cytometric Mock Community and a manual for performing the method of claim 10.

13. Use of the microbial Cytometric Mock Community of claim 1 in standardisation of flow cytometric measurement.

14. A kit comprising the microbial Cytometric Mock Community and a manual for performing the method of claim 11.
